(19) United States
(12) Patent Application Publication
(10) Pub. No.: US 2009/0228049 A1
(43) Pub. Date: Sep. 10, 2009

(54) Connecting Cannulated Bone Screws

(76) Inventor: SangDo Park, Philadelphia, PA (US)

Correspondence Address: SANGDO PARK, 2400 CHESTNUT ST., APT. 311, PHILADELPHIA, PA, 19103, US

(21) Appl. No.: 12/045,001

(22) Filed: Mar. 9, 2008

(51) Int. Cl. A61B 17/56

(52) U.S. Cl. 606/301; 606/60; 606/320

(57) ABSTRACT

A method of bone fixation comprises engagement of internal threads on an implantable object associated with the bone at one side of the fracture with the external threads on the shaft of another implantable object associated with the bone at the opposite side of the fracture. The preferred embodiment comprises two cannulated screws of different diameters which associate with the bones at the opposite sides of the fracture via their screw heads contacting the bone surfaces, and the screws are screwed into the bones manually or drilled towards each other and engage by means of internal threads of the larger-diameter cannulated screw and the external threads of the smaller-diameter cannulated screw. The engagement of the two cannulated screws is aided by a guiding mechanism.

### BACKGROUND OF THE INVENTION

1. Technical Field

This method of bone fracture fixation is of relevance to the field of orthopaedic surgery.

2. Description of the Prior Art

There are several ways to achieve bone fracture fixation in orthopaedic surgery. The simplest is the use of a lag screw. It consists of a head and a shaft which is partially threaded only distally while the proximal portion of the shaft immediately adjacent to the head is smooth. It works by engaging the threaded portion of the shaft into the bone at the far side of the fracture and compressing this against the bone at the near side of the fracture using the head of the screw. However, there are some limitations to this technique. One limitation is that the lag screw cannot cross more than one fracture line and thus is ineffective against fractures with fracture lines that run roughly parallel. And another limitation is that the lag screw is dependent on the quality of screw thread purchase on the bone at the far side of the fracture. Poor bone quality means poor fixation. Thus in these two instances, a more invasive and time-consuming option such as plate fixation is used.

Through the current invention, the above problems with the lag screws are circumvented. The current invention would allow fixation through multiple fracture lines that are roughly parallel and is not dependent on the quality of screw purchase into the bone. Rather, the screw thread purchase involving the internal threads of a larger-diameter cannulated screw and the external threads of a smaller-diameter cannulated screw, which are in effect screwed into each other, will provide a stronger construct of bone fracture fixation than the lag screw and the ability to traverse multiple fracture lines.

### BRIEF SUMMARY OF THE INVENTION

This method of bone fracture fixation seeks to provide a rigid construct which is effective and offers a means to circumvent the limitations of the commonly used lag screw by utilizing engagement of internal threads on an implantable object associated with the bone at one side of the fracture with the external threads on the shaft of another implantable object associated with the bone at the opposite side of the fracture. The preferred embodiment comprises two cannulated screws of different diameters which associate with the bones at the opposite sides of the fracture via their screw heads contacting the bone surfaces, and the screws are screwed into the bones manually or drilled towards each other and engage by means of internal threads of the larger-diameter cannulated screw and the external threads of the smaller-diameter cannulated screw. The engagement of the two cannulated screws is aided by a guiding mechanism involving a guide pin which guides the trajectory of both screws into the bones and toward each other so that their threads may engage.

### BRIEF DESCRIPTION OF THE DRAWINGS

For a fuller understanding of the nature and objects of the invention, reference should be had to the following detailed description taken in conjunction with the accompanying drawings in which:

FIG. 1 is a cross sectional view of the larger-diameter cannulated screw

FIG. 2 is a cross sectional view of the smaller-diameter cannulated screw

FIG. 3 is a view of the guide pin

FIG. 4 is a cross sectional view of the engagement of the cannulated screws across the fracture site

FIG. 5 is an exterior view of the engagement of the cannulated screws

FIG. 6 is a cross sectional view the larger-diameter cannulated screw and the smaller-diameter cannulated screw using an alternative embodiment

FIG. 7 is a view of the guiding mechanism to be used with an alternative embodiment

FIG. 8 is a cross sectional view of the engagement of the cannulated screws across the fracture site using an alternative embodiment

FIG. 9 is a view of disrupted ankle syndesmosis

FIG. 10 is a view of disrupted ankle syndesmosis fixed with current invention

### DETAILED DESCRIPTION OF THE PREFERRED EMBODIMENTS

FIG. 1 is a cross-sectional view of the larger-diameter cannulated screw with internal threads 4 demonstrating head 1, centrally located proximal bore 2, and the larger distal bore 3 which is in continuity with the proximal bore 2. The larger-diameter cannulated screw may possess external threads 5 along the entire length of the screw shaft as demonstrated in FIG. 1 or partially along the length of the screw shaft.

FIG. 2 is a cross-sectional view of the smaller-diameter cannulated screw with external threads 7 demonstrating head 6 and a centrally located bore 8. The smaller-diameter cannulated screw is sized so that the external threads 7 thread onto and engage the internal internal threads 4 of the larger-diameter cannulated screw. The diameter of bore 8 of the smaller-diameter cannulated screw is equal to the diameter of proximal bore 2 of the larger cannulated screw. The external threads 7 of the smaller-diameter cannulated screw may be present along the entire length of the shaft as demonstrated in FIG. 2 or partially along the length of the shaft.

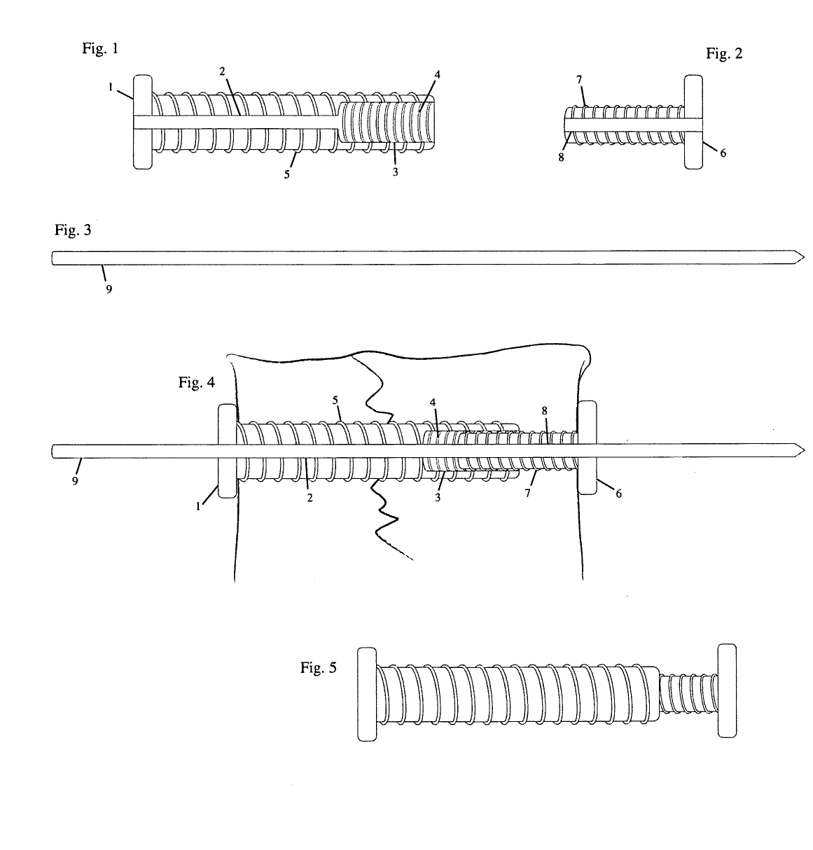

FIG. 3 shows the guide pin 9 which guides the trajectory of the larger- and smaller-diameter cannulated screws into the bones and aids in their engagement. The guide pin 9 allows for appropriate fitting into both the proximal bore 2 of the larger-diameter cannulated screw and the bore 8 of the smaller-diameter cannulated screw. The appropriate fit is defined as the guide pin having a diameter slightly less than the diameter of the bores such that the cannulated screws are free to move along the length of the guide pin with minimal toggling around the guide pin.

FIG. 4 shows the engagement of the screws aided by the guide pin 9.

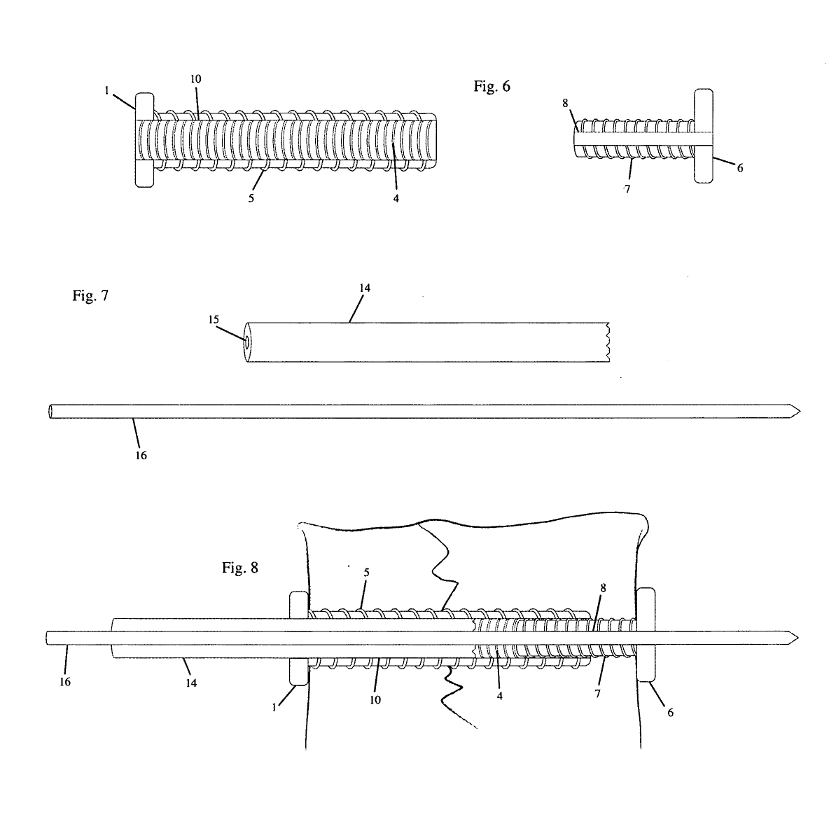

An alternative embodiment of bone fracture fixation is demonstrated in FIGS. 6, 7, and 8. In this alternative embodiment, the larger-diameter cannulated screw has one central bore 10 and internal threads 4. The guiding mechanism is demonstrated in FIG. 7. The cannulated guide pin 14 has a central bore 15 to allow for a guide pin 16 to fit appropriately so that the guide pin 16 can slide along the length of the cannulated guide pin 14 with minimal toggle. FIG. 8 demonstrates the engagement of the cannulated screws across the fracture site using the alternative embodiment.

### EXAMPLE OF USE

Please reference FIG. 4 for this example. The guide pin 9 is drilled into the bone across the fracture site and comes out at the opposite side of the bone. The larger cannulated screw is inserted onto the guide pin 9 and is screwed manually or drilled into the bone until the head 1 contacts the surface of the bone. The appropriate fit of the guide pin 9 within the proximal bore 2 of the larger cannulated screw guides the trajectory of the screw into the bone along the path of the guide pin. Subsequently, the smaller cannulated screw of appropriate length is chosen. Its length must be less than the difference between the width of bone traversed by the guide pin and the length of proximal bore 2 of the larger cannulated screw, but greater than the difference between the width of bone traversed by the guide pin and the length of the larger cannulated screw. The smaller cannulated screw is inserted over the guide pin end which comes out the opposite side of the bone and is screwed manually or drilled into the bone. Its external threads 7 will engage the internal threads 4 of the larger cannulated screw (i.e., the smaller cannulated screw is screwed into the shaft of the larger cannulated screw), and the smaller cannulated screw is inserted until the head 6 contacts the surface of the bone and is tightened. The guide pin 9 is then removed from the bone.

In the alternative embodiment, please reference FIG. 8. The alternative embodiment facilitates choosing the appropriate length of the smaller-diameter cannulated screw. The cannulated guide pin 14 is drilled into the bone. The larger-diameter cannulated screw is inserted onto the cannulated guide pin and is manually screwed or drilled into the bone following the trajectory of the cannulated guide pin into the bone. The guide pin 16 is inserted into the bore 15 of the cannulated guide pin 16 and is drilled into the bone across the fracture and comes out at the opposite side of the fracture. The smaller-diameter cannulated screw need only be of length greater than the difference between the width of the bone and the length of the larger-diameter cannulated screw. It is inserted onto the guide pin 16 which comes out the other side of the bone and is screwed manually or drilled toward the larger-diameter cannulated screw until the internal threads 4 and external threads 7 start to engage, at which point, the cannulated guide pin 14 can be backed out and removed from the bone. After the head 6 of the smaller-diameter cannulated screw contacts the bone and is tightened, the guide pin 16 is removed.

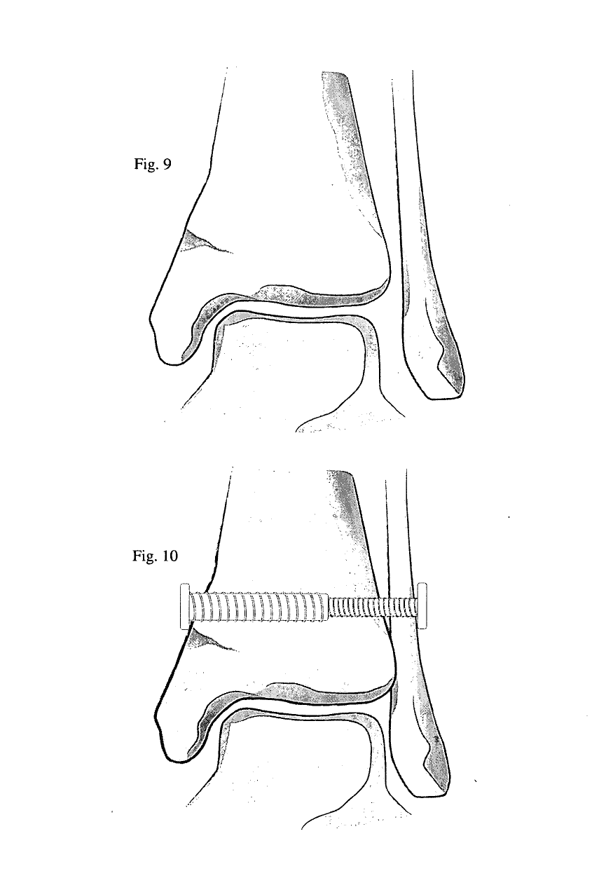

In another alternative embodiment, please reference FIG. 9 which shows a syndesmotic injury of the ankle in which the distal tibio-fibular joint is disrupted. There is no fracture of a bone per se, but the separation of these two bones can be brought together by the current invention as shown in FIG. 10.

Thus a method for bone fracture fixation has been shown and described above. It will be apparent that many changes, modifications, variations, and other uses and applications are possible and contemplated, and all such changes, modifications, variations, and other uses and applications which do not depart from the spirit and scope of the invention are deemed to be covered by the invention as is described in the Claims section.

I claim:

1. A method of bone fracture fixation comprising engagement of internal threads on an implantable object associated with the bone at one side of the fracture with the external threads on the shaft of another implantable object associated with the bone at the opposite side of the fracture.

2. The method of bone fracture fixation as claimed in claim 1 comprises two cannulated screws of different shaft diameters which associate with the bones at the opposite sides of the fracture via their screw heads contacting the bone surfaces, and the screws are screwed into the bones manually or drilled towards each other and engage by means of internal threads of the larger-diameter cannulated screw and the external threads of the smaller-diameter cannulated screw.

3. The larger-diameter cannulated screw as claimed in claim 2 comprises:
a. external threads which cover the shaft length entirely or partially; and
b. a smaller smooth proximal bore central in its location and in continuity with a larger distal bore, which is threaded to engage the external threads of the smaller-diameter cannulated screw.

4. The smaller-diameter cannulated screw as claimed in claim 2 comprises:
a. external threads which cover the shaft length entirely or partially; and
b. a smooth central bore along its entire length which is of the same diameter as the proximal bore of the larger-diameter cannulated screw.

5. The engagement of the two cannulated screws as claimed in claim 2 is aided by a guiding mechanism.

6. The guiding mechanism as claimed in claim 5 comprises a guide pin which is drilled into the bone across the fracture site to come out at the opposite side of the bone and is of a diameter such that the trajectory of the larger-diameter cannulated screw into the bone is guided by the appropriate fit of its smaller proximal bore onto the guide pin, and the trajectory of the smaller-diameter cannulated screw into the bone from the opposite side of the fracture is guided by the appropriate fit of its bore onto the guide pin.

7. The method of bone fracture fixation as claimed in claim 1 comprises two cannulated screws of different shaft diameters; each cannulated screw comprises one central bore of a constant diameter along its entire length; the bore of the larger-diameter cannulated screw is of a larger diameter than the bore of the smaller-diameter cannulated screw and comprises threads which cover the length of the bore entirely or partially; the two cannulated screws comprise external threads which cover the shaft lengths entirely or partially; the two cannulated screws associate with the bones at the opposite sides of the fracture via their screw heads contacting the bone surfaces; and the two cannulated screws are screwed into the bones manually or drilled towards each other and engage by means of internal threads of the larger-diameter cannulated screw and the external threads of the smaller-diameter cannulated screw.

8. The engagement of the two cannulated screws as claimed in claim 7 is aided by a guiding mechanism comprising:
a. a cannulated guide pin with a central bore which is drilled into the bone, and its bore allows a second guide pin of a smaller diameter and of appropriate fit to slide into the bore and be drilled into the bone across the fracture site to come out at the opposite side of the bone; and
b. the trajectory of the larger-diameter cannulated screw into the bone is guided by the appropriate fit of the bore onto the cannulated guide pin, and the trajectory of the smaller-diameter cannulated screw into the bone from the opposite side of the fracture is guided by the appropriate fit of the bore onto the second guide pin of smaller diameter.
